(19) United States
(12) Patent Application Publication
(10) Pub. No.: US 2007/0112248 A1
(43) Pub. Date: May 17, 2007

(54) Drill unit with camera

(75) Inventors: Susumu Terakawa, Shizuoka (JP); Takashi Sakurai, Shizuoka (JP); Seiji Yamamoto, Shizuoka (JP); Atsuo Miyakawa, Shizuoka (JP); Hideo Hirukawa, Tokyo (JP); Takeo Tanaami, Tokyo (JP)

Correspondence Address: VENABLE LLP, P.O. BOX 34385, WASHINGTON, DC, 20043-9998, US

(73) Assignee: Yokogawa Electric Corporation, Tokyo (JP)

(21) Appl. No.: 11/599,590

(22) Filed: Nov. 15, 2006

(30) Foreign Application Priority Data
Nov. 16, 2005 (JP) JP2005-332038

(51) Int. Cl. A61B 1/00

(52) U.S. Cl. 600/104

(57) ABSTRACT

A drill unit equipped with a camera, a body of which has a camera hole at the center, and two holes formed to the left and right side of the camera hole 4 including a discharge hole 2 for discharging air and water to the drilled portion, and a suction hole 3 for sucking water and air from the drilled portion. A first and a second drill holes are formed at the top and bottom of the camera hole. Illuminating holes are also formed to illuminate the drilled portion. The camera serves to project an image of the drilled portion. The first and the second drilling members are used to fracture the body tissues, to clean the fractured portion with water, and to suck water used for the cleaning so as to be discharged. The body of the drill unit is rotated to change the drilling position for the fracture in the same way for forming the hole led to the affected portion.

### BACKGROUND OF THE INVENTION

1. Field of the Invention

The present invention relates to a drill unit equipped with a camera, which is used to drill a live body to make a hole through which an imaging fiber is inserted for the purpose of observing an object such as an affected area of the body.

2. Description of the Related Art

A system for observing an affected area of a live body using a microscope or a monitor has been put into a practical use. In the system, an imaging fiber is inserted to irradiate the affected area of the body from a leading end thereof for observing such area upon reception of the reflected light ray.

The imaging fiber for the use in such organs as digestive system and urologic system may be introduced from such opening of the body as a mouth to reach the affected area through channels thereof. The imaging fiber for the use in the circulatory organ, for example, may be introduced into the blood vessel from a part of the body that allows the introduction easily to reach the affected area. If the object organ has no opening to allow the introduction of the imaging fiber to reach the affected area, it is necessary to drill the body to make a hole for introducing the imaging fiber to the affected area.

There has been introduced no device that punctures the body to the affected area to guide the imaging fiber smoothly while allowing the observation of such area simultaneously. Generally, incision of the body deep into the affected area, or drilling of the body is performed, and thereafter, an endoscope is set for observation and treatment.

Patent Document 1 (Japanese Unexamined Patent Application Publication No. 8-238215) which discloses an exemplary structure of an endoscope shows an insertion channel structure of a treating tool for an endoscope. The disclosed structure reduces the size of the control unit while allowing sufficient inserting performance of the treating tool and ensuring layout space of the contents. The control unit includes the ocular portion, the lever for curving operations, the suction button, and the air/water button as well as two ports at the lower portion of the control unit for insertion of two forceps.

There are the pipelines, the air/water tube, the erection wire, the light guide cable, and the image guide within an insertion portion for illumination and observation while allowing the treatment of the affected area using the erection wire, the forceps and the like.

The above-structured endoscope is introduced to the affected area through the digestive system or the circulatory organ, or the hole preliminarily punctured. It is not provided with the function for drilling the hole through which the endoscope is introduced to the affected area.

### SUMMARY OF THE INVENTION

Accordingly, it is an object of the present invention to provide a drill unit equipped with a camera that includes a mechanism for selecting a suitable drill in accordance with the tissue either hard or soft, and drilling the hole to the affected area with efficiency and minimum invasion while allowing the observation and treatment in the drilled state thereafter.

According to the embodiment of the invention, a drill unit equipped with a camera is provided with a long column-shaped member which includes a camera hole that allows an insertion of the camera for taking an image of a target to be observed and a drilled portion, at least one drill hole that allows a drilling member to be inserted to a tip end of the drill unit, a discharge hole through which one of air and water is supplied to the drilled portion, a suction hole through which the one of air and water from the drilled portion is sucked to be recovered, and at least one illuminating fiber hole for illuminating an area near the drilled portion. The tip end of the drill unit is inserted into a guide hole formed in a live body. The illuminating fiber illuminates the drilled portion such that the camera is used to observe the drilled portion while driving the drilling member inserted into the drill hole. One of air and water is supplied from the discharge hole to the drilled portion so as to be recovered through the suction hole to further drill the guide hole deep to reach a target to be observed.

In the embodiment, the long column-shaped member is stored within a cylindrical portion so as to be rotatable therein.

In the embodiment, the drilling member is formed as a unit that fractures the drilled portion with a drill.

In the embodiment, the drilling member is formed as one of an ultrasonic disintegrator, a high pressure water atomizer, and a laser source for fracturing the drilled portion using ultrasonic wave, high pressure water and laser light rays.

The drill unit is capable of efficiently forming a hole that leads to a target to be observed yet minimizing the invasion during the drilling. After the hole reaches the target, such unit is allowed to perform the observation and treatment within the hole, thus alleviating the burden caused by the drilling of the body.

### BRIEF DESCRIPTION OF THE DRAWINGS

FIG. 1A is a sectional view taken along line A-A of the drill unit equipped with a camera according to an embodiment of the present invention;

FIG. 1B is a sectional view taken along line B-B of the drill unit equipped with a camera according to the embodiment of the present invention;

FIG. 2 shows transverse cross sections of a drill unit equipped with a camera according to the embodiment of the present invention;

FIG. 3A is a view of an exemplary drill as the drilling member;

FIG. 3B shows exemplary structures of an ultrasonic disintegrator;

FIG. 3C is a view of an exemplary structure of a high pressure water atomizer;

FIG. 4 is a view of a laser disintegrator; and

FIG. 5 is a view showing how the drill unit equipped with a camera is operated.

### DESCRIPTION OF THE PREFERRED EMBODIMENTS

An embodiment of the present invention will be described in detail referring to the drawings.

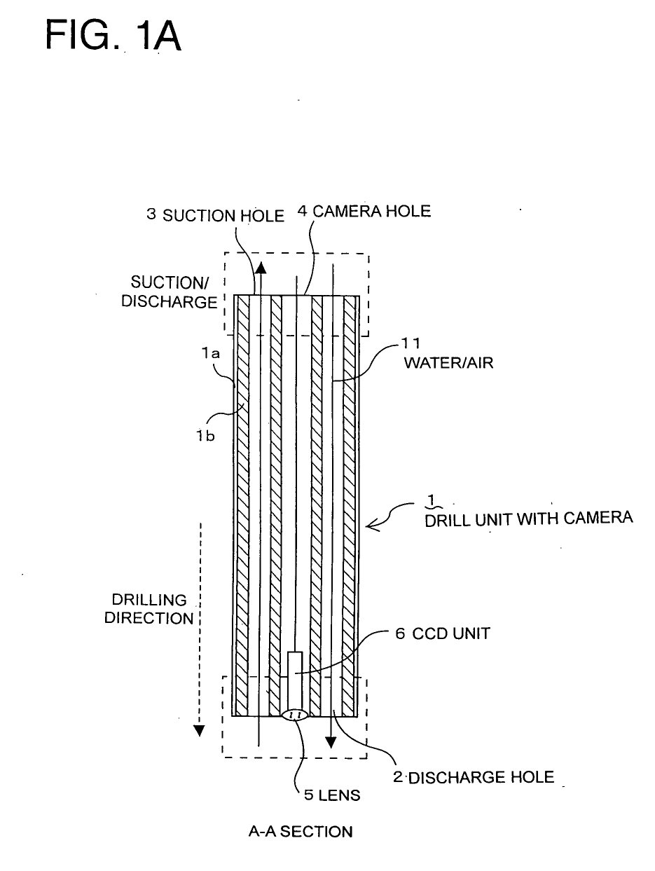

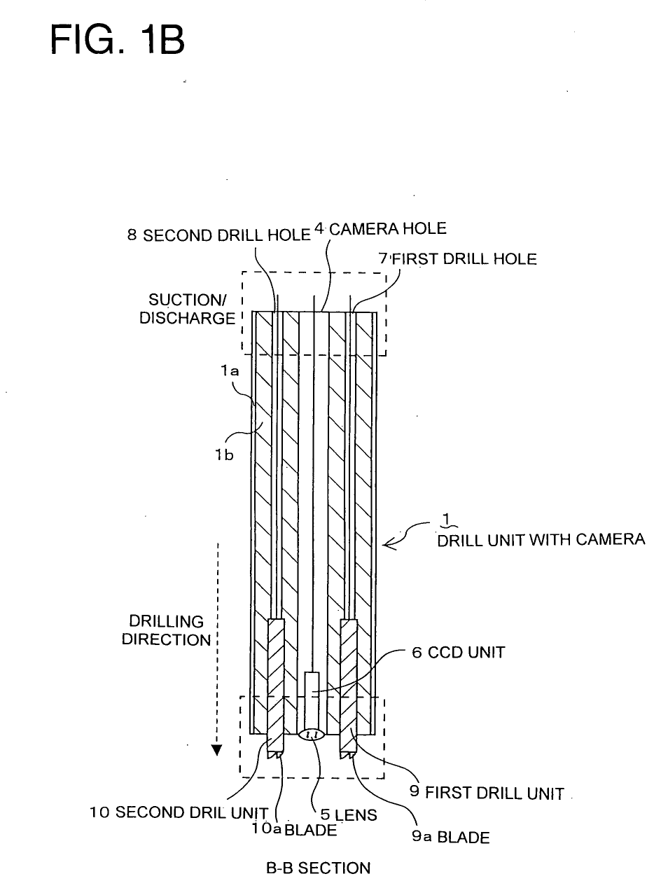

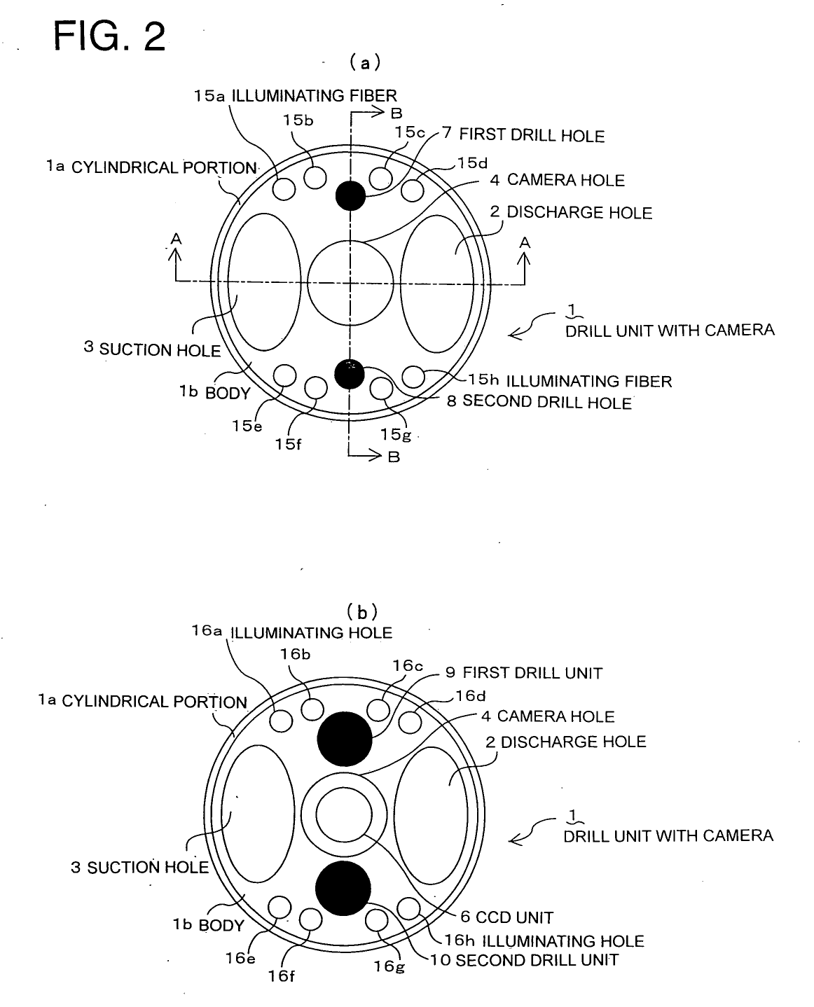

Each of FIGS. 1A and 1B shows a drill unit equipped with a camera according to an embodiment of the present invention. FIGS. 1A and 1B respectively show sectional views taken along lines A-A and B-B of the drill unit shown in FIG. 2. A body 1b of the drill unit formed of a resin material has a long column-like shape. The body 1b is fit with a cylindrical portion 1a as a cover so as to be slidable in an axial direction therein as well as rotatable in a circumferential direction. The rotating angle of the body 1b in the circumferential direction is set at minimum of 90° clockwise and counterclockwise.

A camera hole 4 is formed through the center of the body 1b for accommodating an imaging fiber with a camera. A discharge hole 2 for admitting water or air into the drilled portion, and a suction hole 3 for discharging water or air drawn from the drilled portion are formed to the left and right of the camera hole 4. A first drill hole 7 and a second drill hole 8 are formed at a rotation angle of approximately 90° with respect to the discharge hole 2 and the suction hole 3, respectively.

A first drill unit 9 and a second drill unit 10 are respectively inserted into the first drill hole 7 and the second drill hole 8 such that an organ of the body is fractured by a mechanical element such as blades 9a and 10a at each tip of the drills, a high pressure water atomizer, an ultrasonic disintegrator, or a laser disintegrator.

FIG. 2 shows the drill unit equipped with a camera according to the embodiment of the present invention, wherein FIG. 2A is a view of a transverse cross section of a root portion of the drill unit, and FIG. 2B is a view of a transverse cross section of a leading end portion of the drill unit. A plurality of illuminating holes 16a to 16h are formed outside the first drill hole 7 and the second drill hole 8 such that illuminating fibers 15a to 15h are respectively inserted therein. Each transverse cross section of the suction hole 3 and the discharge hole 2 has an extended oblong shape. A CCD unit 6 is set in the tip portion of the camera hole 4 for taking images of the drilled portion which has been illuminated by the illuminating fibers 15a to 15h.

The first drill unit 9 and the second drill unit 10 protrude from the respective ends of the first drill hole 7 and the second drill hole 8, respectively. It is possible to set the drill in one of those two drill holes. It is also possible to replace the currently used drill with that of different type during the drilling operation. For example, the high pressure water atomizer is used for drilling the soft tissues which contain relatively large number of blood vessels to contact only with the soft tissue without damaging the blood vessel. The ultrasonic disintegrator is then replaced for drilling the hard portion such as bones so as to be fractured.

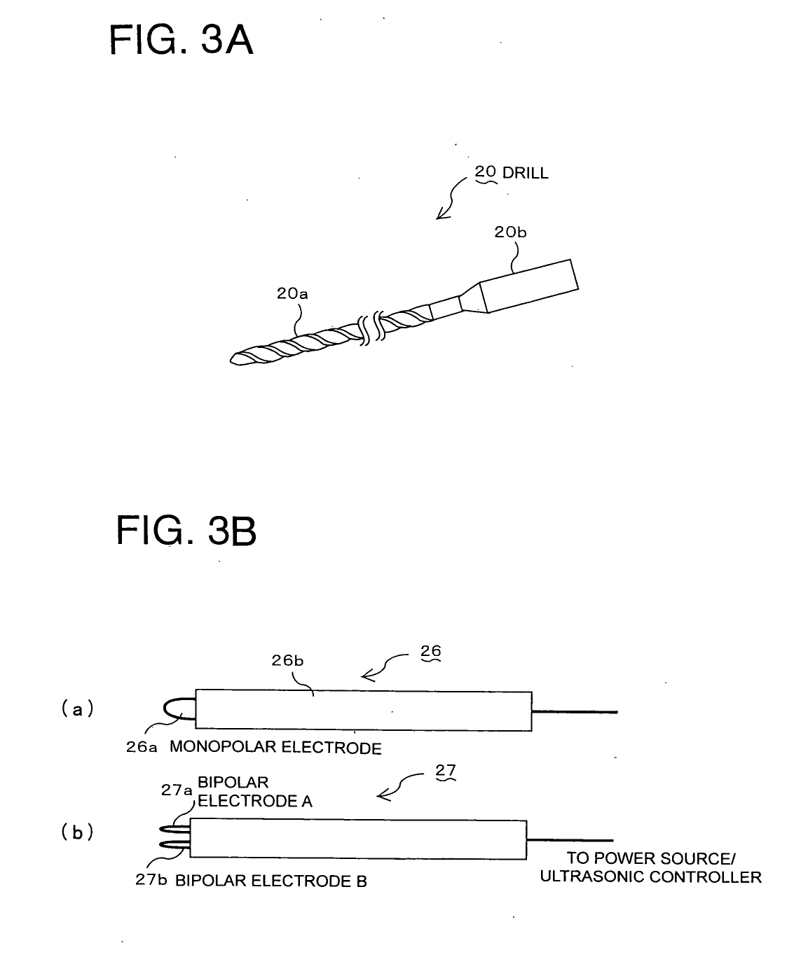

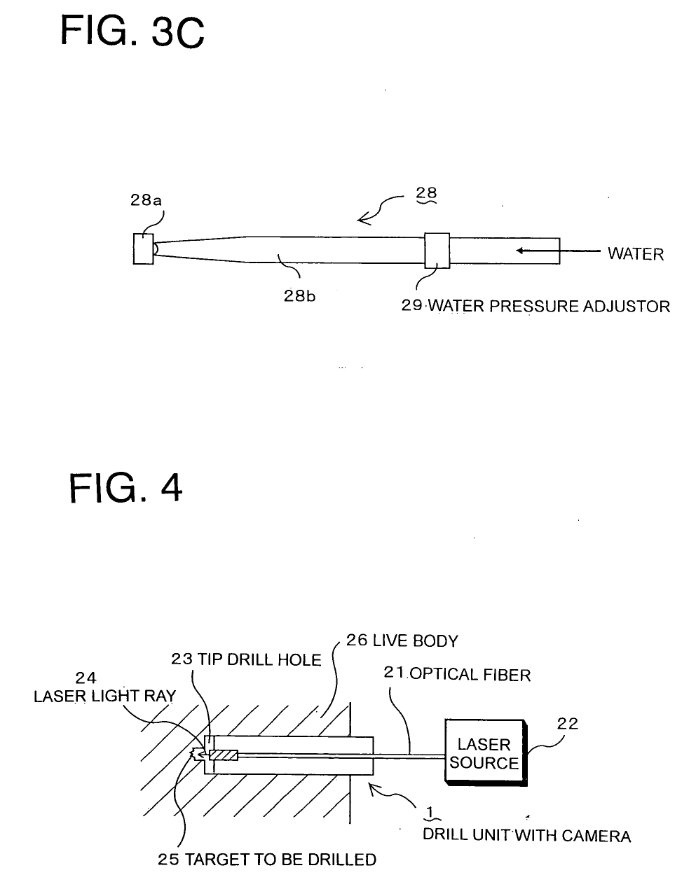

FIG. 3A shows an example of the structure of the drill as the drilling member. A drill 20 has a screw-like blade 20a at its leading end, and has a long root portion 20b. It is inserted into the first drill hole 7 or the second drill hole 8 such that the leading end protrudes from the drill hole. Meanwhile, the root portion 20b of the drill 20 that protrudes from the upper surface of the drill hole is connected to a rotary drive source (output shaft of the motor) for rotatably driving the drill 20 so as to chip the hard tissue of the body.

FIG. 3B shows exemplary structures of the ultrasonic disintegrator as a drilling member, wherein the upper view shows a structure that fractures the body tissue using a monopolar electrode. An ultrasonic disintegrator 26 of monopolar type has a monopolar electrode 26a protruding from a tip end of a handle portion 26b. An electric wire connected to the monopolar electrode 26a is led behind the handle portion 26b. The monopolar electrode 26a is inserted from the upper surface of the first or the second drill hole so as to protrude from the lower surface. A power source and ultrasonic controller (not shown) then is connected to the electric wire connected to the monopolar electrode 26a.

The body tissue opposite the monopolar electrode 26a may be fractured by ultrasonic wave generated by the power source and ultrasonic controller. The ultrasonic wave is transmitted from the monopolar electrode 26a toward the opposite body tissue such that a relatively wide range of the body tissue extended from the monopolar electrode 26a is fractured.

The lower view of FIG. 3B shows an ultrasonic disintegrator that fractures the body tissue using a bipolar electrode. The arrangement of the electrode to the drill hole is the same as that of the monopolar ultrasonic disintegrator except the fracture range of the body tissue. The ultrasonic wave is transmitted from the respective bipolar electrodes to the body tissue. As the ultrasonic wave is hardly transmitted to the peripheral tissue, this makes it sure to fracture only the target tissue. Accordingly, the fracture range may be adjusted while minimizing the invasion. Further, the ultrasonic wave is effective for fracturing the hard tissue like bones.

FIG. 3C is a view of an exemplary structure of a high pressure water atomizer as the drilling member. A high pressure water atomizer 28 allows fracture contact only with the soft tissue such as fat through water atomization without damaging the blood vessel. A water pressure adjuster 29 is provided at the intermediate portion of the handle portion 28b for controlling the water pressure to the one corresponding to the hardness of the body tissue subjected to the fracture contact.

FIG. 4 is a view of an exemplary laser disintegrator as the drilling member. An optical fiber 21 connected to a laser source 22 is inserted into the first or the second drill hole of the drill unit 1 equipped with the camera such that the tip end protrudes into a tip drill hole 23 to transfer the laser light ray to a target 25 to be drilled for proceeding the drilling while fracturing the body tissue.

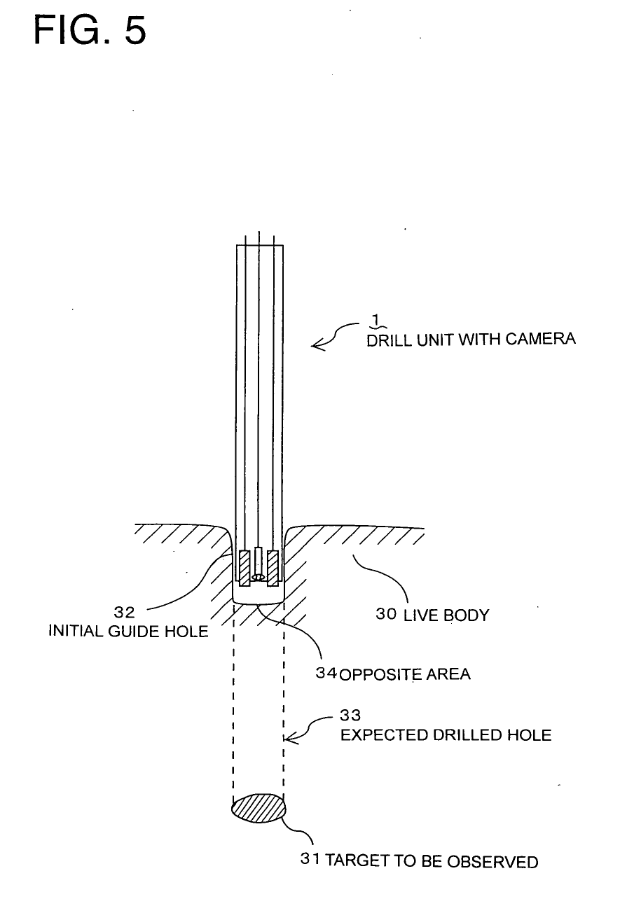

FIG. 5 is a view of the drill unit equipped with the camera according to the embodiment of the present invention. An initial guide hole 32 is formed through a live body 30 for guiding the tip end of the drill unit 1 equipped with the camera. The drill unit 1 equipped with the camera is inserted into the initial guide hole 32 to fracture the body tissues using the ultrasonic disintegrator while observing the image of the tip end portion taken by the camera. As the fracture range is limited to the area opposite the disintegrator, the target range is only fractured, and water is supplied to the fractured portion to be washed. The used water is sucked to be discharged outside. When the fracture of one target area is finished, the drill unit is rotated to further perform the fracture. In this way, an area 34 of the body tissue opposite the drill unit is fractured. This makes it possible to gradually drill an expected hole 33 to reach the target 31 to be observed.

In the aforementioned embodiment, two drill holes are formed. However, one drill hole or more than two drill holes may be formed. A plurality of illuminating holes that allow insertion of the illuminating fibers are formed outside the first and the second drill holes. However, they may be formed around the camera hole located at the center.

In the embodiment, the body may be rotated at 90° clockwise and counterclockwise with respect to the cylindrical portion for drilling all the portions opposite the drill unit. However, four drill holes may be formed at every rotation angle of 90° such that all the area opposite the drill unit may be drilled without increasing the rotating amount of the body.

The drill unit equipped with a camera in the medical field according to the present invention allows drilling of the hole that reaches the affected area of the body while making it possible to observe and treat the affected area.

What is claimed is:

1. A drill unit equipped with a camera comprising a long column-shaped member which includes:
a camera hole that allows an insertion of the camera for taking an image of a target to be observed and a drilled portion;
at least one drill hole that allows a drilling member to be inserted to a tip end of the drill unit;
a discharge hole through which one of air and water is supplied to the drilled portion;
a suction hole through which the one of air and water from the drilled portion is sucked to be recovered; and
at least one illuminating fiber hole for illuminating an area near the drilled portion;
wherein the tip end of the drill unit is inserted into a guide hole formed in a live body;
the illuminating fiber illuminates the drilled portion such that the camera is used to observe the drilled portion while driving the drilling member inserted into the drill hole; and
one of air and water is supplied from the discharge hole to the drilled portion so as to be recovered through the suction hole to further drill the guide hole deep to reach a target to be observed.

2. The drill unit according to claim 1, wherein the long column-shaped member is stored within a cylindrical portion so as to be rotatable therein.

3. The drill unit according to claim 1, wherein the drilling member comprises a unit that fractures the drilled portion with a drill.

4. The drill unit according to claim 1, wherein the drilling member comprises one of an ultrasonic disintegrator, a high pressure water atomizer, and a laser source for fracturing the drilled portion using ultrasonic wave, high pressure water and laser light rays.
